(19) United States
(12) Patent Application Publication
(10) Pub. No.: US 2007/0100352 A1
(43) Pub. Date: May 3, 2007

(54) Cartridge suture anchor delivery device, suture anchor delivery device and associated method

(76) Inventors: Daren L. Deffenbaugh, Winona Lake, IN (US); Hugo A. Pedrozo, Silver Lake, IN (US); Matthew M. Biss, Paris, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/263,359

(22) Filed: Oct. 31, 2005

(51) Int. Cl. A61F 2/00

(52) U.S. Cl. 606/104

(57) ABSTRACT

A suture anchor delivery device for use in performing surgery on tissue of a patient is provided. The suture anchor delivery device includes an applicator and a cartridge removably secured to the applicator. The suture anchor delivery device further includes an anchor operatively associated with the cartridge.

### CROSS-REFERENCE TO RELATED APPLICATIONS

Cross reference is made to the following applications: DEP5604 titled, “SUTURE ANCHOR CARTRIDGE HOLDER, SUTURE ANCHOR CARTRIDGE AND ASSOCIATED METHOD” and DEP5605 titled “MULTIPLE SUTURE ANCHOR DELIVERY DEVICE, SUTURE ANCHOR DELIVERY KIT AND ASSOCIATED METHOD” filed concurrently herewith which are incorporated herein by reference.

### TECHNICAL FIELD OF THE INVENTION

The present invention relates generally to the field of orthopaedics, and more particularly, to an implant for use in arthroplasty.

### BACKGROUND OF THE INVENTION

The skeletal system includes many long bones that extend from the human torso. These long bones include the femur, fibula, tibia, humerus, radius and ulna.

A joint within the human body forms a juncture between two or more bones or other skeletal parts. The ankle, hip, knee, shoulder, elbow and wrist are just a few examples of the multitude of joints found within the body. As should be apparent from the above list of examples of joints, many of the joints permit relative motion between the bones. For example, the motion of sliding, gliding, and hinge or ball and socket movements may be had by a joint. For example, the ankle permits a hinge movement, the knee allows for a combination of gliding and hinge movements and the shoulder and hip permit movement through a ball and socket arrangement.

The joints in the body are stressed or can be damaged in a variety of ways. For example, the gradual wear and tear is imposed on the joints through the continuous use of a joint over the years. The joints that permit motion have cartilage positioned between the bones providing lubrication to the motion and also absorbing some of the forces direct to the joint. Over time, the normal use of a joint may wear down the cartilage and bring the moving bones in a direct contact with each other. In contrast, in normal use, a trauma to a joint, such as the delivery of a large force, from an accident for, example, an automobile accident, may cause considerable damage to the bones, the cartilage or to other connective tissue such as tendons or ligaments.

Arthropathy, a term referring to a disease of the joint, is another way in which a joint may become damaged. Perhaps the best known joint disease is arthritis, which is generally referred to a disease or inflammation of a joint that results in pain, swelling, stiffness, instability, and often deformity.

There are many different forms of arthritis, with osteoarthritis being the most common and resulting from the wear and tear of a cartilage within a joint. Another type of arthritis is osteonecrosis, which is caused by the death of a part of the bone due to loss of blood supply. Other types of arthritis are caused by trauma to the joint while others, such as rheumatoid arthritis, Lupus, and psoriatic arthritis destroy cartilage and are associated with the inflammation of the joint lining.

During the lifetime of a patient, it may be necessary to perform a total shoulder replacement procedure on the patient as a result of, for example, disease or trauma. In a total shoulder replacement procedure, a humeral component having a head portion is utilized to replace the natural head portion of the arm bone or humerus. The humeral component typically has an elongated intramedullary stem which is utilized to secure the humeral component to the patient's humerus. In such a total shoulder replacement procedure, the natural glenoid surface of the scapula is resurfaced or otherwise replaced with a glenoid component that provides a bearing surface for the head portion of the humeral component.

As alluded to above, the need for a shoulder replacement procedure may be created by the presence of any one of a number of conditions. One such condition is the deterioration of the patient's scapula in the area proximate to the glenoid surface as a result of, for example, glenohumeral arthritis. In such a condition, the erosion of the patient's scapula is generally observed posteriorly on the glenoid surface. Such erosion of the scapula renders treatment difficult, if not impossible, with a conventional glenoid prosthesis.

One alternative to implanting a metallic and/or plastic glenoid is the positioning of a biological scaffold in the form of an implant including a biological agent over the natural glenoid to promote healing and regrowth of the natural glenoid. Various biological agents are available for including in the scaffolding to resurface the glenoid. One such product is manufactured from the extra cellular matrix of vertebras. Such an extra cellular matrix is more fully described in U.S. Pat. Nos. 4,902,508; 4,956,178; 5,372,821; and 5,995,110, and incorporated herein in their entireties by reference. One such product utilizing an extra cellular matrix is in the form of a multi-layer sheet derived from swine intestine submucosa (hereinafter referred to as “SIS”), and sold by DePuy Orthopaedics, Inc., Warsaw, Ind., under the trademark Restore®. The Restore® patch is typically secured to the glenoid cavity by sutures which are secured to the glenoid and to the Restore® patch.

The securing of soft tissue and such extra cellular matrix sheets to the tissues is a slow and time-consuming process. Typically, a suture is anchored into the bone with a suture anchor and the suture is then threaded to the soft tissue. Due to the size of the Restore® patch, multiple anchors are preferably utilized with attached sutures to obtain proper securement of the Restore® patch to the glenoid fossa.

Currently, when multiple suture anchors are to be implanted in the body, for example, for use with the extra-cellular matrix patch such as the Restore® patch, the surgeon is required to place the anchors in a specific pattern one anchor at a time. The requirement to place the anchors in a pattern one anchor at a time leaves great room for human inaccuracy in the procedure. This problem is particularly apparent when implanting a glenoid-resurfacing patch. The patch must be properly secured. Sutures that are anchored to bone, for example a glenoid, are typically used. For a glenoid patch, multiple suture anchors are required for proper securement. To secure multiple sutures anchors, the surgeon must first drill and place all anchors one at a time and in a defined pattern orientation. This process is slow and time consuming.

It should be appreciated that the time a patient is in a surgical environment should be minimized. Therefore, this slow and tedious process increases the length of time required for the patient in the surgery room. Further, the surgeon may place the sutures in a pattern that may be less than the ideal pattern due to difficulty in accessing the glenoid fossa.

The present invention is adapted to overcome at least some of the aforementioned problems.

### SUMMARY OF THE INVENTION

The multiple suture anchor delivery system of the present invention allows for accurate placement of multiple suture anchors in a unique array. A preloaded cartridge, for example, may contain a set number of suture anchors in an array designed for ideal placement of the suture anchors. The cartridge may be loaded into an instrument capable of implanting all the anchors at once into the body. The cartridge will then be ejected and discarded, allowing for a new cartridge to be loaded into the instrument.

According to the present invention, the glenoid fossa may be prepared by first drilling a pattern of holes into the glenoid fossa. After the holes are prepared, all the suture anchors may be implanted at once by placing them in the suture anchor cartridge and then into the installation instrument. This procedure allows for simplicity, efficiency and user friendliness to the surgeon.

A preloaded cartridge with a defined orientation for all the suture anchors provided. This preloaded cartridge will slide, for example, over a slot in the main shaft of the cartridge assembly device. The preloaded cartridge will be pressed within the cartridge assembly device and may, for example, be locked into the assembly device. The assembly device may include an anatomically shaped glenoid contact surface and may be placed against the glenoid bone surface. The assembly device may include an internal spring and provide for implanting all the suture anchors into the glenoid cavity at once. The internal spring may be used to return the cartridge to the starting position to permit the cartridge to be easily ejected from the cartridge holder.

According to one embodiment of the present invention, there is provided a suture anchor delivery device for use in performing surgery on tissue of a patient. The suture anchor delivery device includes an applicator and a cartridge removably secured to the applicator. The suture anchor delivery device further includes an anchor operatively associated with the cartridge.

According to another embodiment of the present invention there is provided a suture anchor delivery kit for use in performing surgery on tissue of a patient. The suture anchor delivery kit includes a suture applicator and a cartridge, removably secured to said applicator. The delivery kit also includes an anchor removably secured to said cartridge.

According to a further embodiment of the present invention, there is provided a method for delivering sutures on tissue on a glenoid fossa of a scapula of a patient. The method includes the steps of providing an anchor with attached sutures for attachment to bone and providing a cartridge for receiving the anchor. The method also includes the steps of providing a holder for holding the cartridge and assembling the anchor into the cartridge. The method further includes the step of assembling the cartridge into the holder. The method also includes the steps of positioning the holder over the implant and actuating the holder to advance the cartridge and the anchor through the implant and into the scapula. The method also includes the steps of positioning the implant on the glenoid fossa and using the sutures to secure the implant to the scapula.

According to another embodiment of the present invention there is provided a suture anchor cartridge holder for use with an anchor cartridge in performing surgery on tissue of a patient. The suture anchor cartridge holder includes a applicator that is adapted to receive at least a portion of the anchor cartridge. The suture anchor cartridge holder also includes a guide operatively associated with the applicator and adapted to receive at least a portion of the anchor cartridge.

According to another embodiment of the present invention there is provided a suture anchor cartridge for use in performing surgery. The suture anchor cartridge is adapted for insertion into a suture anchor cartridge holder. The suture anchor cartridge includes a body and an anchor. The anchor is operatively associated with the body.

According to a further embodiment of the present invention, there is provided a method for securing an implant onto a glenoid fossa of a scapula. The method includes the steps of providing an anchor with attached sutures for attachment to bone and providing a cartridge including at least one anchor having at least one suture attached thereto. The method includes the steps of providing a holder for holding the cartridge and assembling the cartridge into the holder. The method further includes the steps of positioning the implant on the glenoid fossa and positioning the holder over the implant The method includes the steps of actuating the holder to advance the cartridge and the anchor into the scapula and using the sutures to secure the implant to the scapula.

According to a further embodiment of the present invention, there is provided a suture anchor delivery device for use in performing surgery on tissue of a patient is provided. The suture anchor delivery device includes an applicator and a plurality of anchors. Each of the anchors is operatively associated with the applicator.

According to another embodiment of the present invention, there is provided a suture anchor delivery kit for use in performing surgery on tissue of a patient. The suture anchor delivery kit includes an applicator and a plurality of anchors. Each of the anchors is operatively associated with the applicator.

According to another embodiment of the present invention, there is provided a method for securing an implant onto bone. The method includes the steps of providing a first anchor with attached sutures for attachment to bone and providing a second anchor with attached sutures for attachment to bone. The method also includes the steps of providing an applicator holding the first and second anchors and assembling the anchor into the applicator. The method also includes the step of positioning the applicator over the implant. The method also includes the step of actuating the applicator to advance the anchor into the bone. The method also includes the steps of positioning the implant on the bone and using the sutures to secure the implant to the bone.

The technical advantages of the present invention include the ability to implant multiple suture anchors at once. For example, according to one aspect of the present invention, a suture anchor cartridge assembly for use in performing surgery on tissues of a patient is provided. The suture anchor cartridge assembly includes a body and a cartridge removably secured to the body. A plurality of anchors is installed into the cartridge. The cartridge assembly is positioned over the implant site and the body and cartridge advance the plurality of anchors simultaneously into the implant site. Thus, the present invention provides for the ability to implant multiple anchors at once.

The technical advantages of the present invention further include the ability to accurately position a plurality of anchors. For example, according to another aspect of the present invention, a suture anchorage cartridge is provided. The suture anchor cartridge is adapted for insertion into a holder. The suture anchor cartridge includes a body and a plurality of pre-positioned and spaced apart anchors that are placed in particular positions in the body. Thus, the present invention provides for accurately positioning a plurality of anchors in a fixed spaced apart relationship.

The technical advantage of the present invention also includes the ability to securely place the anchors. For example, according to yet another aspect of the present invention, a suture anchor cartridge assembly is provided for performing surgery on tissues of a patient. The suture anchor cartridge assembly includes a body, a cartridge and an anchor. The body includes a surface closely conforming to the surface where the anchors are to be placed, as well as a inserting rod that may be positioned with a stop to accurately and securely place the anchor relative to the position in the body where it is to be placed. Thus, the present invention provides for securely placing the anchors in the body.

The technical advantages of the present invention also include the ability to quickly load or refill an anchor cartridge to repair subsequent surgeries. For example, according to yet another aspect of the present invention, a suture anchor cartridge assembly for use in performing surgery on a patient is provided. The cartridge assembly includes a body and a cartridge removably secured to the body. Anchors are operatively associated or positioned on the cartridge. The cartridge may be quickly removed from the body after the anchors have been separated from the cartridge. A second or new cartridge may be easily positioned onto the body, so that a subsequent use of the body may occur. Thus, the present invention provides for the ability to quickly load a cartridge into the applicator.

The technical advantages of the present invention also include the ability to sterilize the cartridge assembly. For example, according to another aspect of the present invention, the suture anchor cartridge assembly may include a body, a cartridge and an anchor, each of which may be sterilizable. The cartridge may be pre-loaded with the anchors with the cartridge and anchor pre-sterilized. The body may include components that are readily disassembled. The body may be resterilizable and be made of sterilizable components that are sterilizable from a commonly available sterilizing technique, such as by an autoclave. Thus, the present invention provides for a suture anchor cartridge assembly that may be sterilized.

The technical advantages of the present invention further include the ability to provide for a disposable cartridge. By providing a disposable cartridge that includes preassembled anchors, the cartridge when spent, may be disposed. Thus, the amount of effort or time by the surgery team may be minimized to merely inserting a new cartridge into a sterile cartridge holder or body. Thus, the present invention provides for a disposable cartridge.

Other technical advantages of the present invention will be readily apparent to one skilled in the art from the following FIGS., descriptions and claims.

### BRIEF DESCRIPTION OF THE DRAWINGS

FIG. 1 is a perspective view of a multiple suture anchor cartridge applicator assembly according to an embodiment of the present invention;

FIG. 1A is a perspective view of a suture anchor cartridge applicator assembly according to another embodiment of the present invention having a solitary anchor;

FIG. 1B is a perspective view of a multiple suture anchor cartridge applicator assembly according to another embodiment of the present invention having two anchors;

FIG. 1C is a perspective view of a multiple suture anchor cartridge applicator assembly according to another embodiment of the present invention having three anchors;

FIG. 2 is an perspective view of a multiple suture anchor cartridge for use in the multiple suture anchor cartridge applicator assembly of FIG. 1;

FIG. 3 is a plan view of a suture anchor for use in the multiple suture anchor cartridge of FIG. 2 in position over the cartridge of FIG. 2;

FIG. 4 is a perspective view of the multiple suture anchor cartridge applicator of the multiple suture anchor cartridge applicator assembly of FIG. 1;

FIG. 5 is a perspective view of the cartridge guide and plunger shaft of the multiple suture anchor cartridge applicator of FIG. 4;

FIG. 5A is a bottom view of the cartridge guide of FIG. 4;

FIG. 5B is a front view of the cartridge guide of FIG. 4;

FIG. 5C is an end view of the cartridge guide of FIG. 4;

FIG. 5D is a perspective view of a multiple suture anchor cartridge applicator assembly according to another embodiment of the present invention having a guide with relief areas;

FIG. 5E is a perspective view of a multiple suture anchor cartridge applicator assembly according to another embodiment of the present invention having a small central guide;

FIG. 5F is a perspective view of the pin for securing the spring of the multiple suture anchor cartridge applicator assembly of FIG. 1;

FIG. 5G is a perspective view of the spring of the multiple suture anchor cartridge applicator assembly of FIG. 1;

FIG. 5H is a perspective view of an alternate biasing member that may be used with the multiple suture anchor cartridge applicator assembly of FIG. 1;

FIG. 5I is a perspective view of a multiple suture anchor cartridge applicator assembly in the expanded position according to an embodiment of the present invention having a guide with relief for over the columns assisting in viewing the glenoid fossa;

FIG. 5J is a perspective view of the multiple suture anchor cartridge applicator assembly of FIG. 5J in the retracted position;

FIG. 6 is a perspective view of the base of the multiple suture anchor cartridge applicator of FIG. 4;

FIG. 7 is a perspective view of the multiple suture anchor cartridge applicator assembly of FIG. 1 showing the cartridge in a partially assembled position;

FIG. 8 is a perspective view of the multiple suture anchor cartridge applicator assembly of FIG. 1 showing the cartridge in a fully assembled position;

FIG. 8A is a perspective view of the multiple suture anchor cartridge applicator assembly of FIG. 1 showing the cartridge in a position as it begins its contact with the glenoid fossa;

FIG. 9 is a perspective view of the multiple suture anchor cartridge applicator assembly of FIG. 1 showing the cartridge in a fully inserted position for engagement with the glenoid;

FIG. 10 is a perspective view of a glenoid patch after the suture anchor cartridge applicator assembly of FIG. 1 has been deployed into the patch;

FIG. 11 is an enlarged exploded plan view of a suture anchor of FIG. 3 for use in the multiple suture anchor cartridge of FIG. 2 in position over the cartridge of FIG. 2;

FIG. 12 is a side view of the suture anchor of FIG. 11;

FIG. 13 is an anterior/posterior view of the multiple suture anchor cartridge applicator assembly of FIG. 1 shown in position above the glenoid prior to utilization of the applicator;

FIG. 14 is an medial/lateral view of the a glenoid patch of FIG. 10 in position over the glenoid after deployment of the sutures by the applicator assembly of FIG. 1;

FIG. 15 is a perspective view of a multiple suture anchor cartridge applicator assembly according to an embodiment of the present invention having a small central guide;

FIG. 16 is a perspective view of a multiple suture anchor cartridge applicator assembly according to an embodiment of the present invention having a guide with relief between the columns for assisting in viewing the glenoid fossa;

FIG. 16A is a perspective view of a multiple suture anchor cartridge applicator assembly in the expanded position according to an embodiment of the present invention having a guide with relief for over the columns assisting in viewing the glenoid fossa;

FIG. 16B is a perspective view of the multiple suture anchor cartridge applicator assembly of FIG. 16A in the retracted position;

FIG. 17 is a plan view of a kit for use in performing total shoulder arthroplasty in accordance with another embodiment of the present invention;

FIG. 18 is a flow chart of a method for performing total shoulder arthroplasty in accordance with another embodiment of the present invention;

FIG. 19 is a flow chart of another method for performing total shoulder arthroplasty in accordance with another embodiment of the present invention; and

FIG. 20 is a flow chart of another method for performing total shoulder arthroplasty in accordance with another embodiment of the present invention.

Corresponding reference characters indicate corresponding parts throughout the several views. Like reference characters tend to indicate like parts throughout the several views.

### DETAILED DESCRIPTION OF THE INVENTION

Embodiments of the present invention and the advantages thereof are best understood by referring to the following descriptions and drawings, wherein like numerals are used for like and corresponding parts of the drawings.

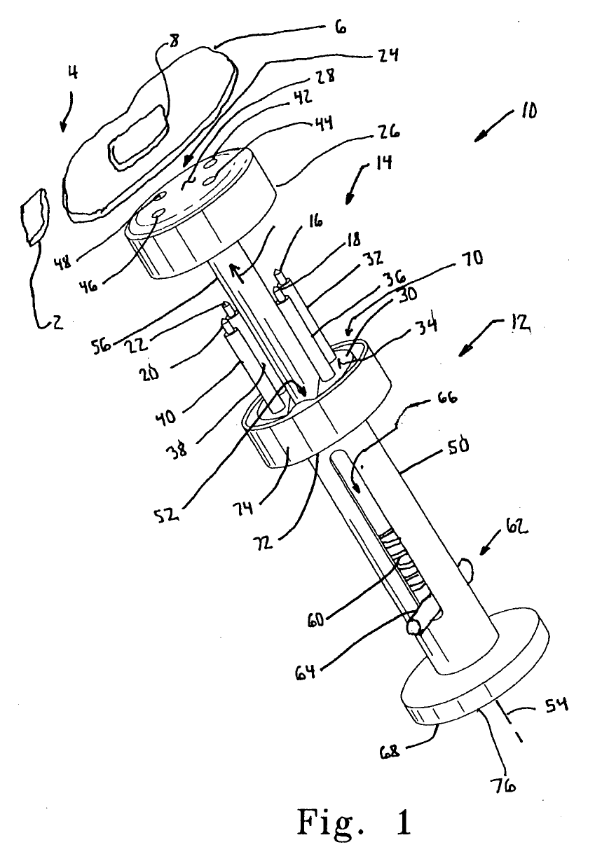

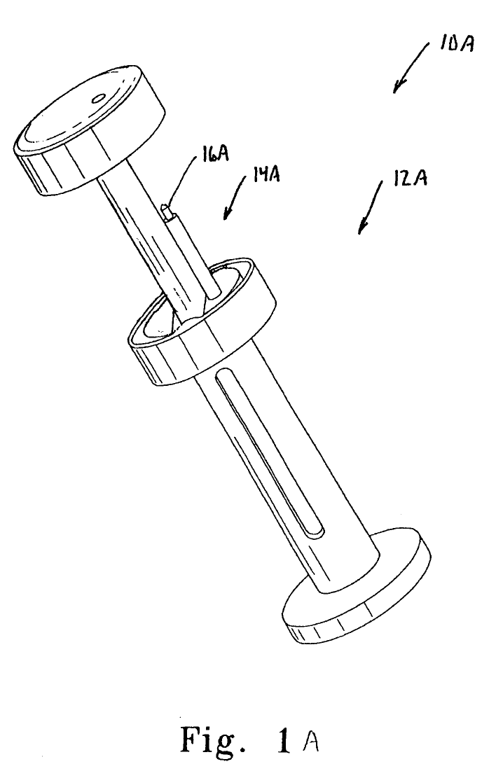

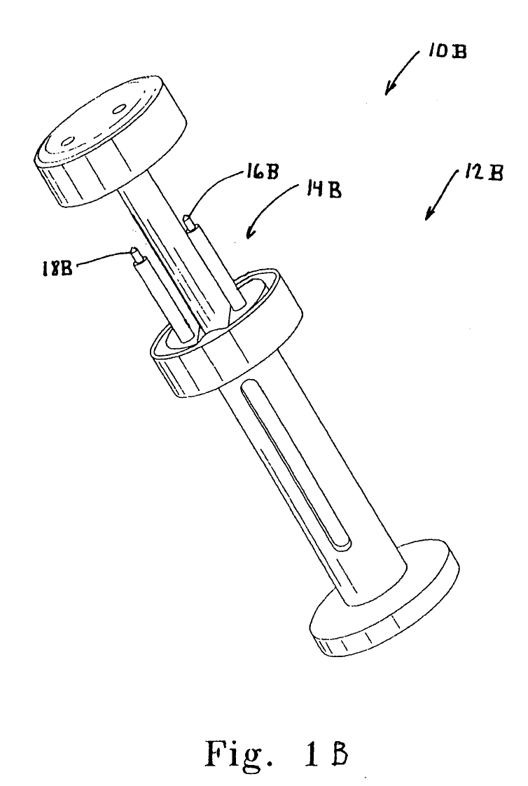

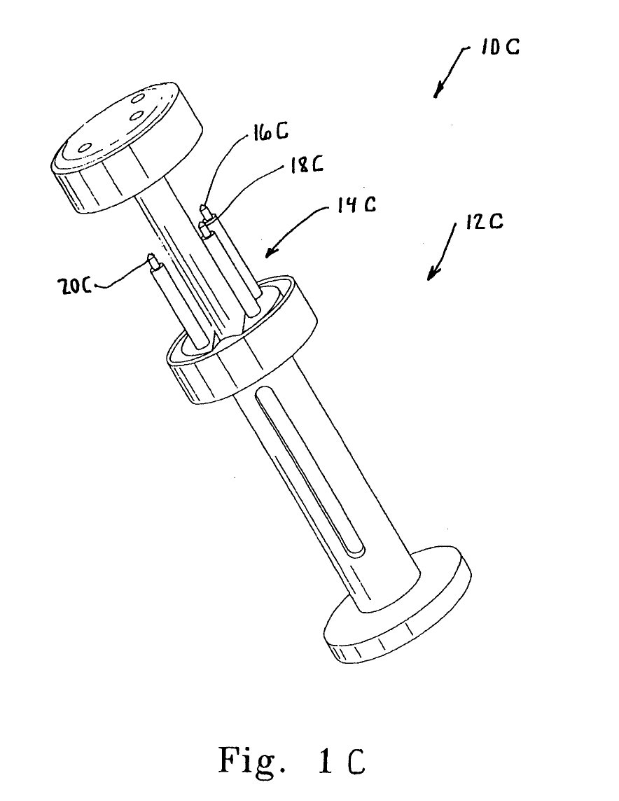

According to the present invention, referring now to FIG. 1, a suture anchor delivery device 10 is shown. The suture anchor delivery device 10 is adapted for use in performing surgery on tissue 2 of patient 4. The suture anchor delivery device 10 includes an applicator 12 and a cartridge 14. The cartridge 14 is removably secured to the applicator 12. An anchor 16 is removably associated with the cartridge 14. The suture anchor delivery device 10 of FIG. 1 may be utilized to secure a solitary anchor 16 or may be used for multiple anchors. It should be appreciated that the use for a single anchor with the use of a cartridge may have value, such as easy anchor replacement or accurate anchor placement with the delivery device.

For example, and referring to FIG. 1A, a suture anchor delivery device 10A is shown. The suture anchor delivery device 10A includes an applicator 12A and a cartridge 14A. The cartridge 14A is removably secured to the applicator 12A. An anchor, for example, anchor 16A, is operatively associated with the cartridge 14A and, may as is shown in FIG. 1A, may be removably secured to the cartridge 14A. The suture anchor delivery device 10A of FIG. 1A is advantageous in that through the use of a cartridge, the anchor may be quickly and reliably removed and positioned on the applicator 10A. Further, the applicator 10A may include locating features such that the anchor may be accurately positioned in the patient 4, both in anatomical position and in its depth.

According to another embodiment of the present invention, and referring now to FIG. 1B, it should be appreciated that the suture anchor delivery device of the present invention may be utilized with multiple anchors. When the suture anchor delivery device is used with multiple anchors, advantages such as the accurate position of the first anchor with respect to the second anchor, as well as, the ability to insert both anchors simultaneously, may be accomplished with the present invention. The suture anchor delivery device 10B of FIG. 1B includes an applicator 12B and a cartridge 14B removably secured to the applicator 12B. The suture anchor delivery device 10B further includes a first anchor 16B. The suture anchor delivery device 10B also includes a second anchor 18B spaced from the first anchor 16B.

Referring now to FIG. 1C, yet another embodiment of the present invention is shown as suture anchor delivery device 10C. The suture anchor delivery device 10C is similar to the suture anchor delivery device 10 of FIG. 1, except that the suture anchor delivery device 10C includes three spaced apart anchors. For example, the suture anchor delivery device 10C includes an applicator 12C and a cartridge 14C. The cartridge 14C is removably secured to the applicator 12B. The suture anchor delivery device 10C includes a first anchor 16C operatively associated with the cartridge 14C. The suture anchor delivery device 10C further includes a second anchor 18C that is also operatively associated with the cartridge 14C and spaced from the first anchor 16C. The suture anchor delivery device 10 further includes a third anchor 20C that is operatively associated with the cartridge 14C and spaced from the first anchor 16C and the second anchor 18C.

Referring again to FIG. 1, the suture anchor delivery device 10 of FIG. 1 includes a second anchor 18, a third anchor 20, and a fourth anchor 22. The first anchor 16, the second anchor 18, the third anchor 20, and the fourth anchor 22 are spaced apart from each other and may form any pattern that is optimal for the securing of an implant 6.

The implant 6 may be any implant that may be at least partially secured to the patient by a suture secured to a suture anchor. For example, and as is shown in FIG. 1, the first anchor 16, the second anchor 18, the third anchor 20, and the fourth anchor 22 may be spaced from each other in a rectangular pattern.

It should be appreciated that the anchors 16, 18, 20 and 22 may be spaced apart in any pre-selected pattern. Such a pattern may be a geometric pattern different that a rectangular, for example, a square, a rhombus, a parallelogram, a trapezoid. It should also be appreciated that the number of anchors may be other than 1, 2 or 4. If the number of anchors is 3, 5 or greater than 5, many different pre-selected patterns may be chosen. Such patters may be geometric or non-geometric. For example, such geometric patterns may include a triangle, for example, a right triangle, an isosceles triangle. For example, such geometric patterns may include a pentagon, a hexagon etc. Oval, elliptical and round patterns may also be used.

Suture anchor delivery device 10 of FIG. 1, may include a portion 24 of the applicator 12, which is used to cooperate with the implant 6 or an implant patient site 8 of the patient 4, such as a glenoid fossa.

The suture anchor delivery device 10 may further include a guide 26 which may be operatively associated with the applicator 12 and with the cartridge 14. The guide 26 may be utilized to guide or direct the anchors 16, 18, 20, and 22 as they are inserted in bone, for example, glenoid 8 of the patient 4. The implant 6 is then attached to the glenoid using the anchored sutures. The guide 26 may alternatively be utilized to guide or direct the anchors 16, 18, 20, and 22 as they are inserted through the implant 6 and then into the glenoid 8 of the patient 4. The guide 26 may, as is shown in FIG. 1, include the portion 24 for cooperation with the patient.

For example, the guide 26 may include the portion 24 in the form of a closely conforming surface 28 having a shape similar to that of the glenoid fossa 8 of the patient, where the anchors are to be installed.

The guide 26 may be utilized to guide the anchors 16, 18, 20 and 22 in any suitable manner. For example, and as is shown in FIG. 1, the guide 26 serves as a guide or bushing plate for positioning the anchors by being in cooperation with portions of the cartridge 14 that support the anchors.

The guide 26 may be used to assist in guiding the anchor 16 in any suitable manner. For example, and as is shown in FIG. 1, the cartridge 14 is used to assist in the guide 26 in positioning the first anchor 16. For example, and as is shown in FIG. 1, the cartridge 14 includes a base 30 to which a generally cylindrically shaped first column 32 extends normally or perpendicularly from top face 34 of the base 30. The first anchor 16 is secured to the first column 32. Similarly, a second column 34 extends normally from top face 34 of the base 30 and is used to support second anchor 18. A third cylindrical column 38 extends normally from top face 34 of the base 30 and is used to support the third anchor 20. Further, a fourth column 40 having a generally cylindrical shape extends normally from top face 34 of the base 30 and is used to secure fourth anchor 22.

The guide 26, as is shown in FIG. 1, includes openings, for example, first opening 42, which is sized to receive the external cylindrical periphery of the first column 32. Similarly, the guide 26 further includes a second opening 44 sized to receive the second column 36. Likewise, the guide 26 includes a third opening 46 for receiving the third column 38. The guide 26 further includes a fourth opening 48 for receiving the fourth column 40. The columns 32, 36, 38 and 40 cooperate with the openings 42, 44, 46 and 48, to provide for an accurate and steady positioning of the first anchor 16, the second anchor 18, the third anchor 20, and the fourth anchor 22, as they are inserted into the implant 6 and into the bone 8.

As shown in FIG. 1, the guide 26 is slidably connected to the applicator 12. By permitting the guide 26 to slide relative to the applicator 12, the anchors 16, 18, 20 and 22 are permitted to move into their respective openings 42, 44, 46 and 48 of the guide 26. The sliding motion of the guide 26 relative to the applicator 12 may occur in any suitable fashion.

For example, and as is shown in FIG. 1, the applicator 12 includes a body 50 defining a longitudinal opening 52 extending centrally along longitudinal centerline 54 of the applicator 12. A plunger shaft 56 is centrally positioned in opening 52 along centerline 54 and fixably attached to the guide 26. The plunger shaft 56 is adapted to slidably fit in the longitudinal opening 52 of the body 50.

The plunger shaft 56 may, as is shown in FIG. 1, be biased outwardly in the direction of arrow 58, such that the guide 26 is biased to be separated from the cartridge 14. The biasing of the plunger shaft 56 in the direction of arrow 58 may be accomplished by any suitable manner. For example, the applicator 12 may include a biasing member 60, which is used to urge the plunger shaft 56 and attach to guide 26 in the direction of arrow 58 away from the body 50. Biasing member 60 may be in the form of a resilient member. For example, the resilient member 60 may be, for example, a spring or a rubberized material. As shown in FIG. 1, the biasing member 60 is in the form of a helical spring that is fitted within the longitudinal opening 52 of the body 50.

The plunger shaft 56 may be restrained such that a portion of the plunger shaft 56 remains in contact with the body 50 of the applicator 12. For example, the plunger shaft 56 may include a transverse opening 62 for receiving a pin 64. The pin 64 may extend from the transverse opening 62 into a pair of diametrically opposed longitudinally elongated slots or openings 66 formed in the hollow body 50. The pin 64 is permitted to move longitudinally within the slots 66. The pin 64 restrains a portion of the plunger shaft 56 within the longitudinal opening 52 of the body 50.

The pin 64 cooperating with the slot 66 in the body 50 limits the rotational movement of the plunger shaft 56 with respect to the body 50. This limit on the rotational movement provides that the columns 32, 36, 38 and 40, which support the anchors 16, 18, 20 and 22, are in rotational alignment with their respective openings 42, 44, 46 and 48. Thus, the cartridge 14 may extend into the openings 42, 44, 46 and 48 to assist in the release of the anchors 16, 18, 20 and 22 into the glenoid fossa 8.

As shown in FIG. 1, the body 50 may include a circular base 68 for providing a surface for holding and urging the applicator 12 in the direction of arrow 58, such that the anchors 16, 18, 20 and 22 may be driven into the glenoid fossa 8. The body 50 of the applicator 12 may further include a pocket 70 for receiving the cartridge 14. The pocket 70 may be formed by, for example, a circular flange 72 extending outwardly from the body 50 of the applicator 12. The flange 72 with a cylindrical ring that may extend axially from the outer periphery of the flange 72 in the direction of arrow 58. The flange 72 and the ring 74 form the pocket 70 for receiving the cartridge 14. The pocket 70 preferably has a shape compatible with receiving the cartridge 14 and may closely conform to the cartridge 14.

When utilizing the applicator 12, the closely conforming surface 28 of the guide 26 is placed against glenoid fossa 8 and the outer surface 76 of the body base 68 is pushed open to advance the body 50 in the direction of arrow 58 along longitudinal axis 54 until the anchors 16, 18, 20, and 22 begin to engage with the glenoid fossa 8. Then, the body 50 of the applicator 12 is held and a tool, for example, a mallet is used to strike the outer surface 76 of the body base 68 to drive or engage the anchors into the glenoid fossa 8.

The suture anchor delivery device 10 of the present invention may be made of any suitable durable material that is sterilizable and compatible with the human body. For example, the guide 26 and the plunger shaft 56 may be made of any suitable durable material and may be integral with each other or may be permanently secured to each other. The guide 26 and the plunger shaft 56 may be made of, for example, a durable plastic, a composite, or a metal. If made of a metal, the guide 26 and the plunger shaft 56 may be of, for example, a cobalt chromium alloy, a titanium alloy, or a stainless steel alloy.

The body 50 of the applicator 12 of the suture anchor delivery device 10 may likewise be made of any suitable durable material and may be made of, for example, a plastic, a metal, or a composite material. If made of a metal, the body 50 may be made of, for example, a cobalt chromium alloy, a stainless steel alloy, or a titanium alloy.

The pin 64 and the spring 60 may be made of any suitable material, for example, a metal. If made of a metal, the pin 64 and the spring 60 may be made of, for example, a cobalt chromium alloy or a stainless steel alloy.

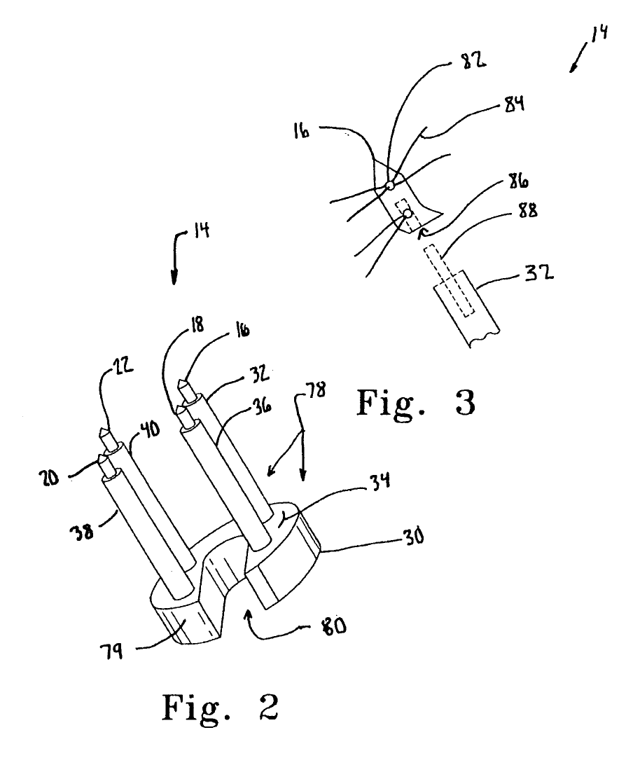

Referring now to FIG. 2, the suture anchor cartridge 14 is shown in greater detail. The suture anchor cartridge 14 is for use in performing surgery. The suture anchor cartridge is adapted for insertion into the suture anchorage cartridge bolder 12 of FIG. 1. The suture anchorage cartridge 14 includes a body 78 and a first anchor 16 operatively associated with the body 78. The body 78 may be integral or may, as is shown in FIG. 2, include the base 30, as well as first column 32 extending perpendicularly from top face 34 of the base 30. It should be appreciated that the cartridge 14 may be adapted for a solitary anchor or for a multiple array of anchors.

For example in FIG. 2, four spaced apart anchors are shown representing a generally rectangular pattern. In addition to the first anchor 16, which extends from first column 32, the second anchor 18 extends from second column 36. Similarly, third anchor 20 extends from third column 38 and fourth anchor 22 extends from fourth column 40. The columns 32, 36, 38 and 40 may be integral with the base 30 or may be fixably secured to the base 30. The anchors 16, 18, 20 and 22 are removably secured to the columns 32, 34, 38 and 40, respectively, by any suitable manner.

The cartridge 14 may, as shown in FIG. 2, include a central opening or slot 80 for receiving the plunger shaft 56 of the applicator 12 (see FIG. 1). The central opening or slot 80 has a width and depth sufficient to provide clearance to the shaft 56 of the applicator 12.

The cartridge 14 may be made of any suitable durable material and may, for example, be made of a combination of plastic components, composite components or metals. Since the cartridge 14 may be a disposable item, the use of low cost materials such as plastics or composites may be preferred. The base 30 may, for example, may be made of a durable plastic, for example, polyethylene. The columns 32, 36, 38 and 40 may likewise be made of a plastic or due to their small size, may be made of a more high-strength material, for example, a metal. If made of a metal, the columns may be made of, for example, cobalt chromium alloy, stainless alloy, or titanium alloy. The anchors, for example, the first anchor 16, the second anchor 18, the third anchor 20 and the fourth anchor 22, may, for example, be made of a metal. For example, the anchors may be made of a stainless steel.

The cartridge 14 is adapted for insertion into the pocket 70 of the applicator 12 of FIG. 1. Since the cartridge 14 is adapted to be fitted into applicator 12, the base 30 of the cartridge 14 includes an external periphery 79, which preferably mates with the pocket 70 of the applicator 12.

Referring now to FIG. 3, an anchor, for example, first anchor 16 is shown in greater detail. The first anchor 16 may include a plurality of suture holes 82 for receiving sutures 84. The sutures 84 are preferably pre-fitted to the suture holes 82 of the anchor 16 and are deployed with the anchor 16 when the anchor delivery device 10 is utilized. The first anchor 16 is fitted to the first column 32 in any suitable manner. For example, and as is shown in FIG. 3, the first anchor 16 includes an internal cavity 86 for receiving stem 88 extending from column 32. The stem is slidably fitted into the cavity 86 such that the anchor 16 may be released from the stem 88 and the cartridge 14 after it has deployed into the glenoid fossa 8.

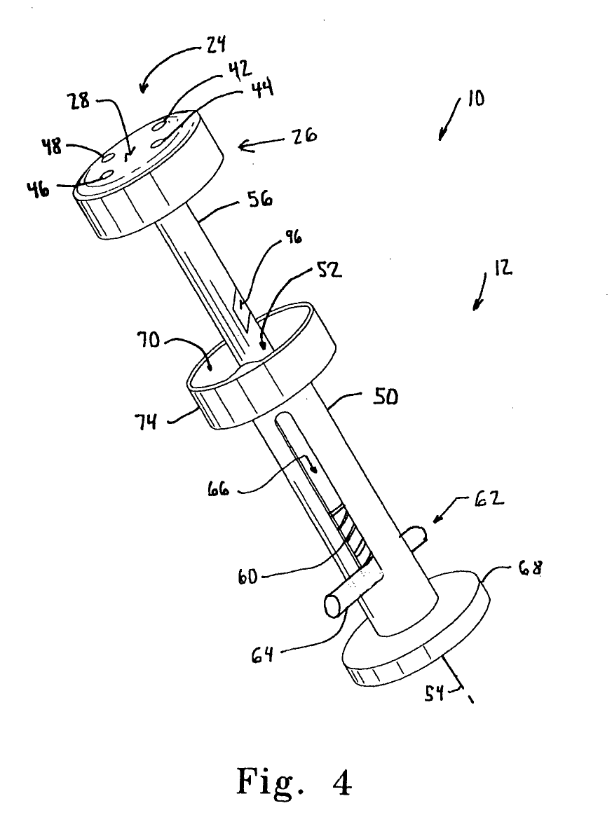

Referring now to FIG. 4, the suture anchor cartridge holder or applicator 12 of the device 10 is shown in greater detail. The applicator 12, as is shown in FIG. 4, has the plunger shaft 56 extended to receive the cartridge 14 into the applicator 12. The suture anchor cartridge holder or applicator 12 is for use with the anchor cartridge such as anchor cartridge 14 of FIG. 2 for use in performing surgery on tissue of a patient. The suture anchor cartridge holder 12 includes the body 50. The body 50 is adapted to receive a portion of the anchor cartridge 14 (see FIG. 2). The suture anchor cartridge holder 12 further includes the guide 26. The guide 26 is operatively associated with the body 50 and is adapted to receive a portion of the anchor cartridge 14 (see FIG. 2).

The applicator 12 may provide such that the guide 26, as is shown in FIG. 4, includes the portion 24 for cooperation with the tissues of the patient. The guide 26 may include the surface 28 for closely conforming to the tissues of the patient. As shown in FIG. 4, the applicator 12 may be provided such that the guide 26 is slidably connected to the body 50. For example, the plunger shaft 56 extending from the guide 26 slidably fits in longitudinal opening 52 of the body 50.

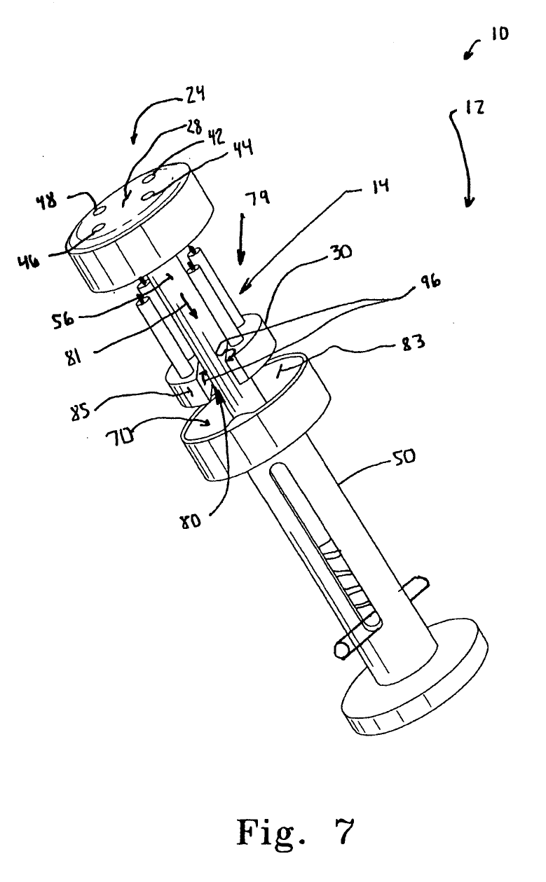

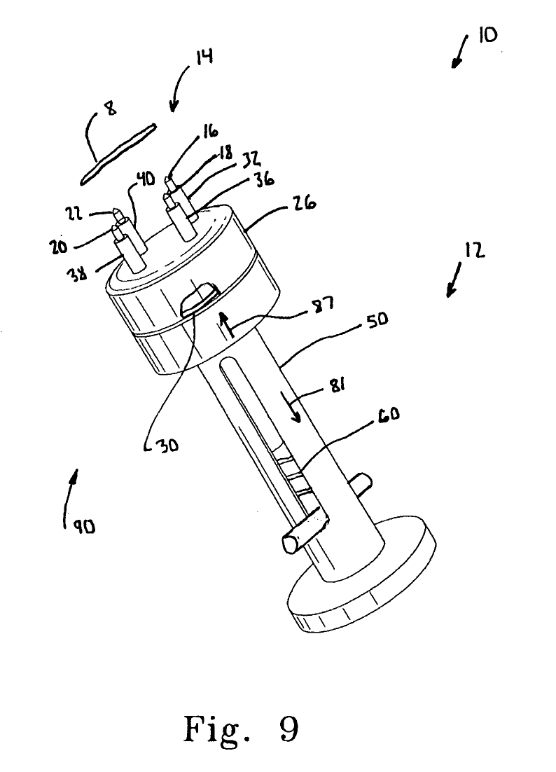

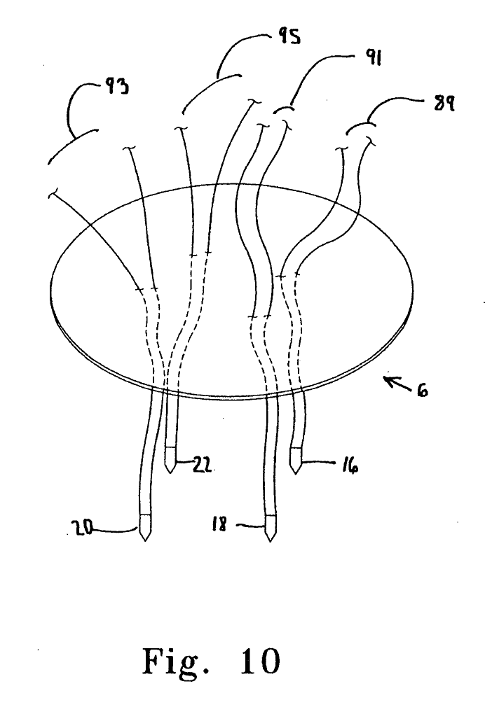

The applicator 12 may be provided, as is shown in FIG. 4, such that the portion 24 engages with the tissue of the patient and the guide 26 and the body 50 are configured to have a first relationship 90, as shown in FIG. 9, in which a portion of the cartridge 14 extends beyond the surface 28 of the guide. The guide 26 and the body 50 also have the second relationship 92, as shown in FIG. 7, in which the cartridge 14 is positioned below surface 28 of the guide 26.

As shown in FIG. 4, the applicator 12 includes biasing mean 60. The biasing mean 60 urges the cartridge in the direction of arrow 58. As shown in FIG. 4, the biasing mean 60 is in the form of a spring. As shown in FIG. 4, the applicator 12 includes the guide 26. The guide 26 includes an internal wall defining an opening, for example, first opening 42, for the passage of at least a portion of cartridge 14, for example, the first anchor 16 and the first column 32, through at least a portion of the first opening 42 (see FIG. 1).

As shown in FIG. 4, the body 50 of the applicator 12 includes the pocket 70, which forms a cavity in the body 50. The pocket 70 is adapted for receiving at least a portion of the cartridge 14.

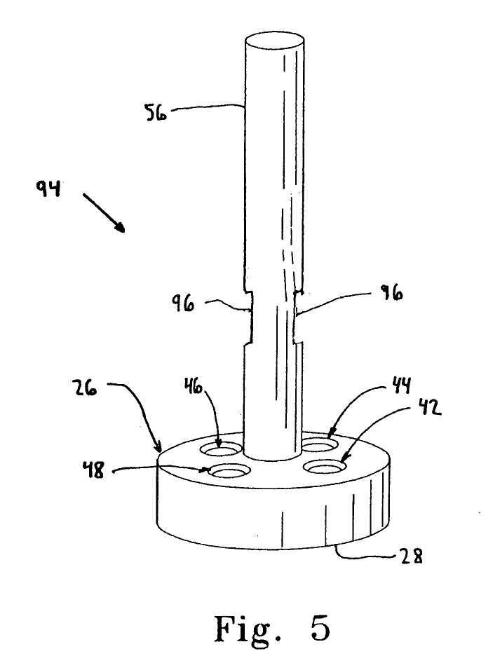

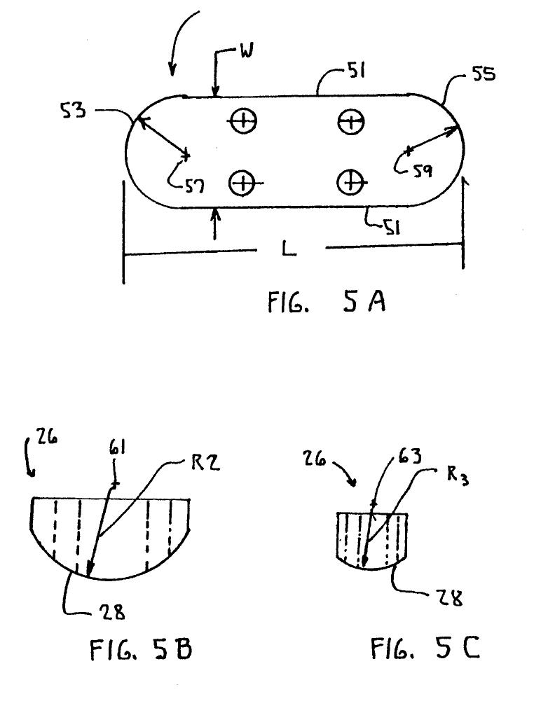

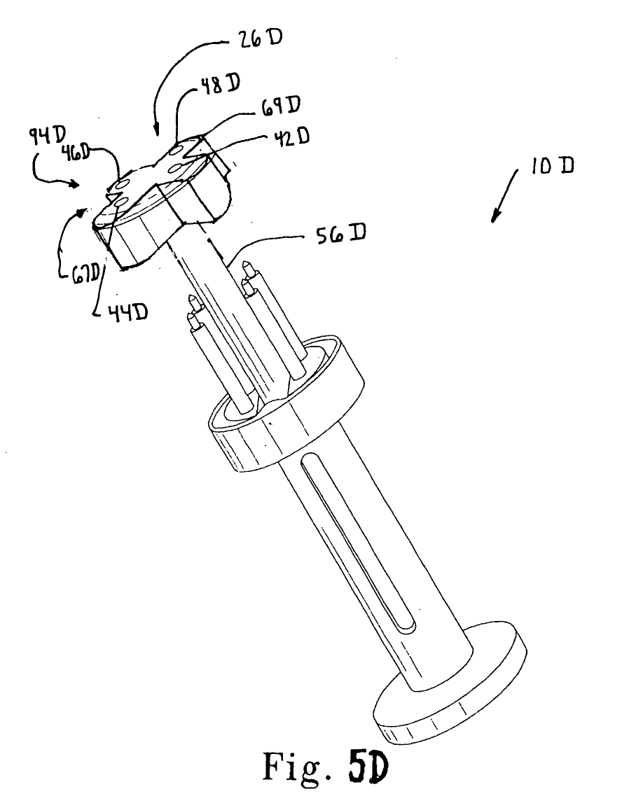

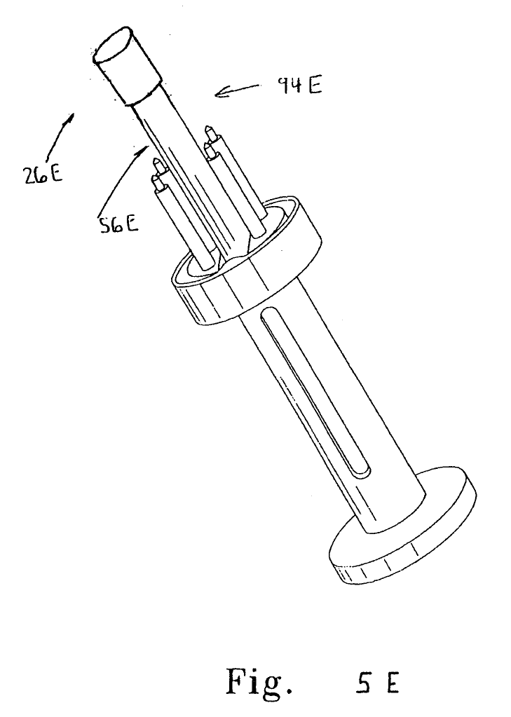

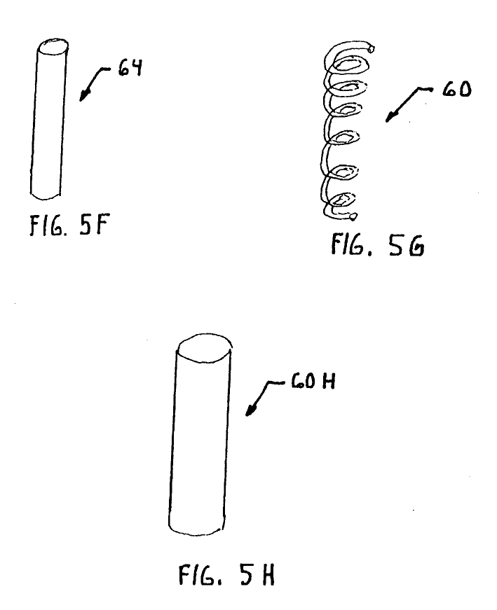

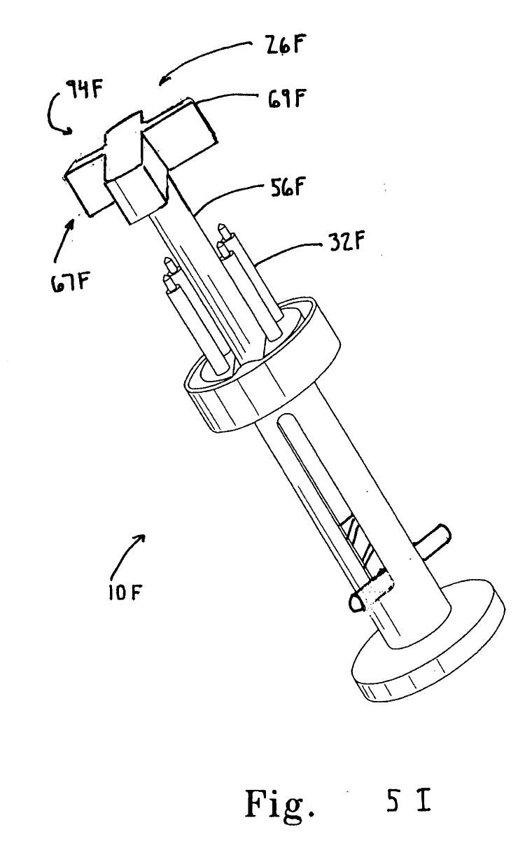

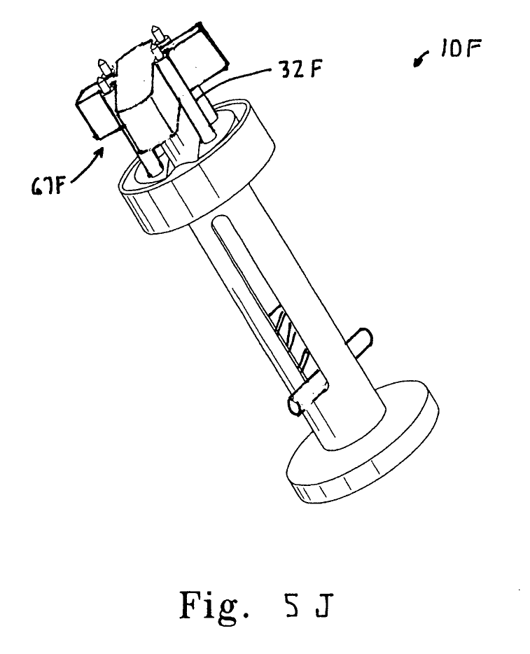

Referring now to FIG. 5, plunger/guide component 94 is shown. The plunger/guide component 94 includes the guide 26 and the integral plunger shaft 56, which extends from the guide 26. The guide 26 includes the first opening 42, the second opening 44, the third opening 46, and the fourth opening 48. The guide 26 further includes closely conforming surface 28 for positioning on the glenoid fossa 8. The plunger shaft 56 may include, as is shown in FIG. 5, spaced apart parallel flats 96 for receiving the cartridge 14 (see FIG. 2).

Referring now to FIGS. 5A, 5B and 5C, the closely conforming surface 28 of the guide 26 is shown in greater detail. The closely conforming surface 28 of the guide 26 includes a periphery 98, which preferably mates with the periphery of the glenoid fossa 8.

The periphery 98, may, as is shown in FIG. 5A, include spaced apart parallel faces 51, which are separated by a distance W. The periphery 98, further defined by arcuate end portions 53 and 55. The first arcuate end portion 53 is defined by radius R extending from origin 57. Similarly, the second arcuate portion 55 is defined by radius R extending from origin 59. The arcuate end portions 53 and 55 are spaced apart by the length L.

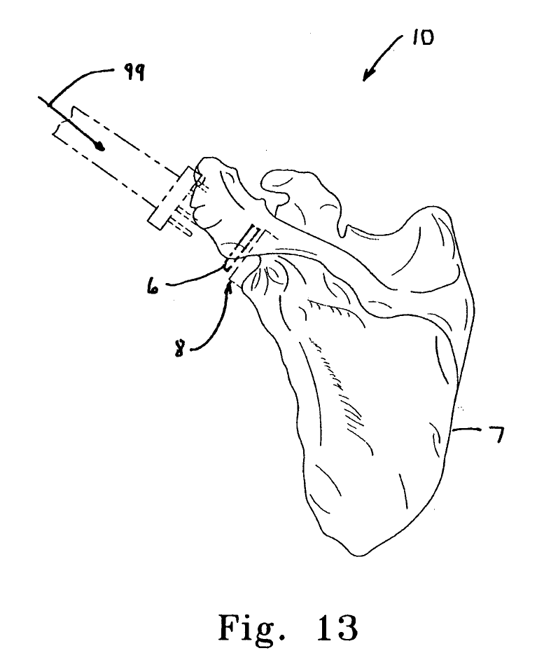

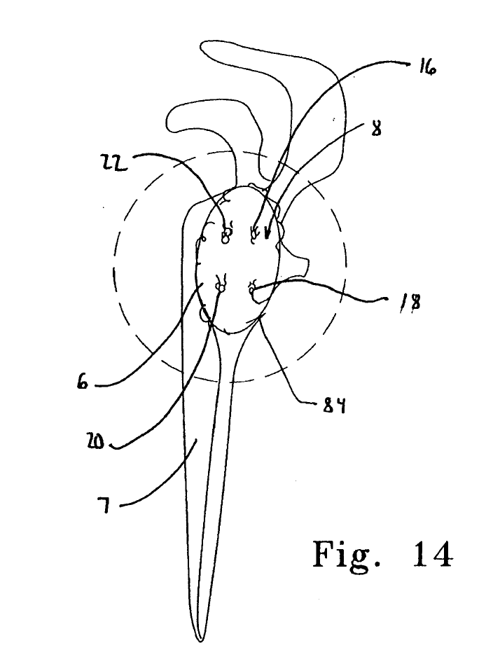

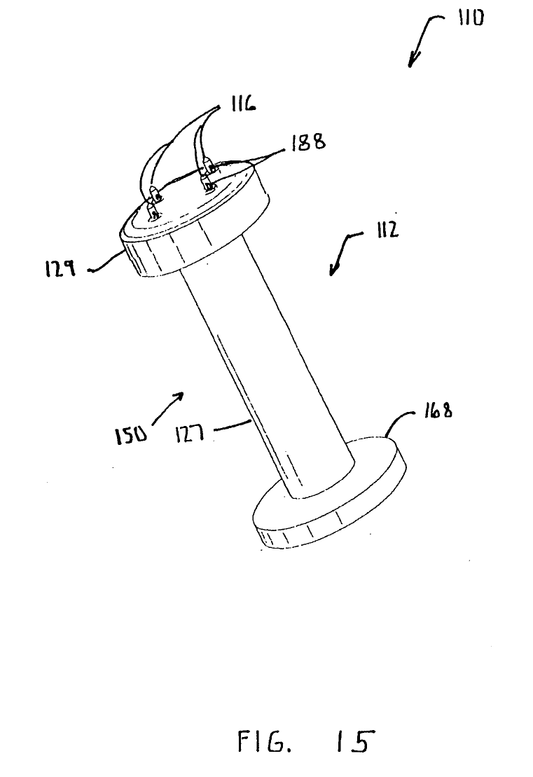

Referring now to FIGS. 5B and 5C, the closely conforming surface 28 of the guide 26 is adapted to conform to the natural glenoid fossa 8 of the scapula 7 (see FIG. 13).

For example, and referring now to FIG. 5B, the closely conforming surface 28 is defined by radius R₂ extending from origin 61. Similarly, in the opposed plane and referring now to FIG. 5C, the closely conforming surface 28 may be defined by radius R₃ extending from origin 63.

Referring now to FIG. 5D, the suture anchor delivery device of the present invention may be in the form of suture anchor delivery device 10D. The delivery device 10D includes a plunger guide component 94D that is slightly different than the plunger guide component 94 of FIG. 5. For example, and as is shown in FIG. 5D, the plunger guide component 94D includes a plunger shaft 56D similar to the plunger shaft 56 of FIG. 5. The plunger guide component 94D of the delivery device 10D includes a guide 26D, which is slightly different from the guide 26D of the plunger guide component 94 of FIG. 5. The guide 26D includes relief portions 67D positioned between lobes 69D surrounding the openings 42D, 44D, 46D and 48D formed in the guide 26. Relief portions 67D assist in the visibility of the glenoid fossa when positioning the suture anchor delivery device 10D against the glenoid fossa of the patient.

Referring now to FIG. 5F, the pin 64 is shown. The pin 64 may have a simple cylindrical shape and is adapted to fit into the transverse opening 62 of the plunger shaft 56 (see FIG. 1). The pin 64 may be made of any suitable durable material and, for example, may be made of a metal. If made of a metal, the pin 64 may be made of, for example, a cobalt chromium alloy, a stainless steel alloy, or a titanium alloy.

Referring now to FIG. 5G, the spring 60 of the suture anchor delivery device 10 is shown in greater detail. The spring 60 may be a simple cylindrical helical spring and may be made of any suitable durable material. The spring 60 may be made of spring steel provided the steel is made of a sterilizable material.

Referring now to FIG. 5H, an alternate biasing member for use in a suture anchor delivery device of the present invention is shown as rubber biasing member 60H. The rubber biasing member 60H may be in the form of a cylindrical natural rubber or synthetic rubber member. The synthetic rubber member 60F preferably has a cylindrical shape similar to the spring 60 of FIG. 5G and may be used to replace the spring 60, provided that the rubber biasing member 60F has a similar spring constant.

Referring now to FIG. 5I, the suture anchor delivery device of the present invention may be in the form of suture anchor delivery device 10F. The delivery device 10F includes a plunger guide component 94F that is slightly different than the plunger guide component 94 of FIG. 5. For example, and as is shown in FIG. 5F in its relaxed extended position, the plunger guide component 94F includes a plunger shaft 56F similar to the plunger shaft 56 of FIG. 5. The plunger guide component 94F of the delivery device 10F includes a guide 26F, which is slightly different from the guide 26F of the plunger guide component 94 of FIG. 5. The guide 26F includes relief portions 67F positioned in alignment with columns 32F. Lobes 69F are formed in the guide 26F between the relief portions 67F. Relief portions 67F assist in the visibility of the glenoid fossa when positioning the suture anchor delivery device 10F against the glenoid fossa of the patient.

Referring now to FIG. 5J, the suture anchor delivery device 10F of FIG. 5I is shown in its contracted position. In this position the columns 32F are show in position in the relief portions 67F.

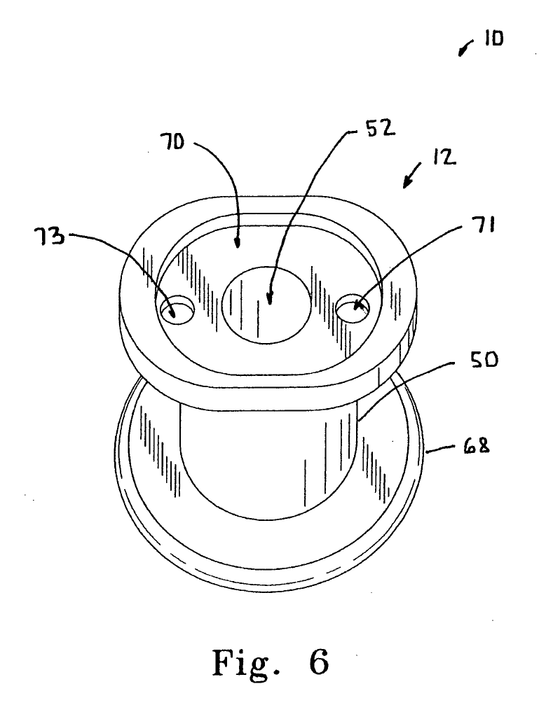

Referring not to FIG. 6, the body 50 of the applicator 12 of FIG. 4 is shown in greater detail. The body 50 includes the body base 68 extending from a first end of the body 50 and the pocket 70 formed in the opposed end of the body 50. The body 50 defines the central longitudinal opening 52 formed in the body 50. The body 50 defines a first ejection hole 71 and a spaced apart second ejection hole 73. The ejection holes 71 and 73 are used to assist in ejecting the cartridge 14 from the body 50 after the cartridge 14 has been used.

Referring now to FIGS. 7, 8, 8A and 9, the utilization of the suture anchor delivery device 10 is shown. Referring first to FIG. 7, the suture anchor cartridge applicator 12 is shown in the extended or open position, also described as the first position, for installing the cartridge 14. The cartridge 14 is inserted in the direction of arrow 79 with the flats 96 formed on the plunger shaft 56 of the applicator 12 in alignment with slot 80 formed in the base 30 of the cartridge 14. The cartridge 14 is advanced in the direction of arrow 79 until the base 30 of the cartridge 14 is fully seated against the plunger shaft 56. Cartridge 14 is then advanced axially in the direction of arrow 81 advancing the base 30 of the cartridge 14 into the pocket 70 formed in the body 50 of the applicator 12. It should be appreciated that internal periphery 83 of ring 74 is slightly larger than external periphery 85 of the base 30 of the cartridge 14.

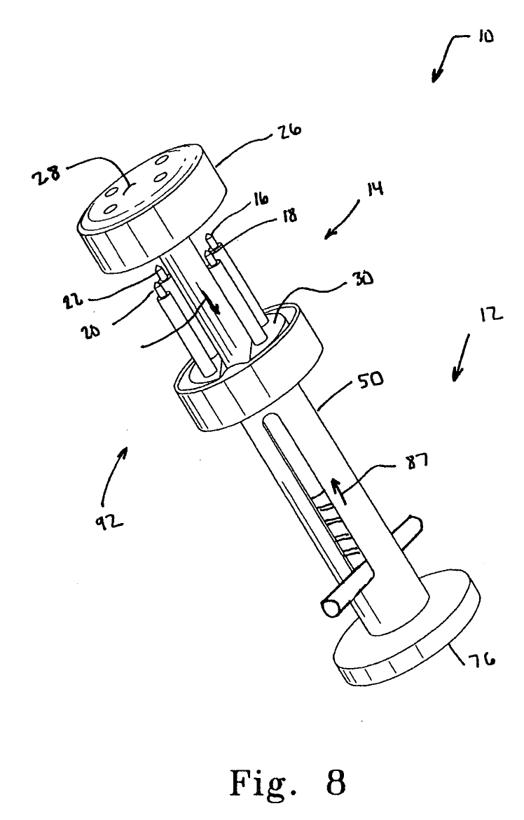

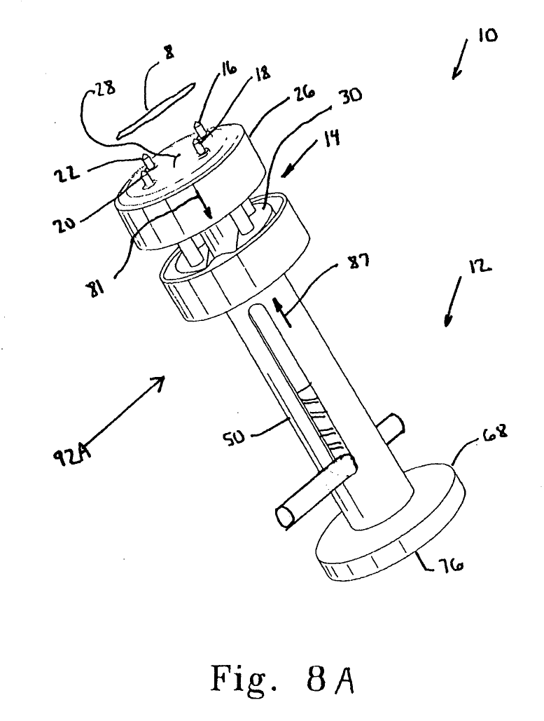

Referring now to FIG. 8, suture anchor delivery device 10 is shown in the second position 92. In the second position 92, the cartridge 14 is advanced in the direction of arrow 81 until the base 30 of the cartridge 14 has fully seated against the body 50 of the applicator 12.

While the suture anchor delivery device 10 is in the second position 92, as is shown in FIG. 8, the suture anchor delivery device 10 is positioned against the anatomy, for example the glenoid fossa, of the patient with the closely conforming surface 28 of the guide 26 in position against the glenoid fossa. Once the anchor delivery device 10 is in position against the glenoid fossa, the body 50 and the cartridge 14 are advanced in the direction of arrow 87, advancing the suture anchors 16, 18, 20 and 22 toward the glenoid fossa.

Referring now to FIG. 8A, the suture anchor delivery device 10 is shown positioned with the body 50 and the cartridge 14 advanced in the direction of arrow 87 to third position 92A until the suture anchors 16, 18, 20 and 22 are in alignment with the closely conforming surface 28 of the guide 26. At this point, the body 50 may not be easily advanced in the direction of arrow 87. At this point, it may be necessary to strike the surface 76 of the base 68 of the body 50 with an instrument, for example, a mallet (not shown), to fully secure the anchors 16, 18, 20 and 22 into the glenoid fossa.

Referring now to FIG. 9, the suture anchor delivery device 10 is shown with the cartridge fully extended in the direction of arrow 87, such that the suture anchors 16, 18, 20 and 22 are fully seated into the glenoid fossa. It should be appreciated that less of the columns, 32, 36, 38 and 40 then shown in FIG. 9 may, in fact, be positioned above the surface 28. As long as a portion of the columns is exposed, the anchors can be deployed. At this point, the suture anchor delivery device 10 may be removed from the glenoid fossa in the direction of arrow 81. The biasing member or spring 60 assists in the movement of the body 50 in the direction of arrow 81 away from the guide 26.

It should be appreciated that the suture anchors 16, 18, 20 and 22 are removed from the columns 32, 36, 38 and 40, such that the cartridge 14 now only includes the cartridge base 30 as well as the columns 32, 36, 38 and 40. The cartridge 14 may either be sterilized and refilled with new suture anchors or discarded. For simplicity, the cartridge 14 may be discarded.

Referring now to FIG. 10, the implant 6 is shown with the suture anchors deployed in bone, for example, the glenoid, below the implant 6. The implant may be placed over the glenoid after the suture anchors are deployed in bone, or the implant may be placed over the glenoid and the suture anchors may be deployed through the implant 6 and then into the glenoid. For example, and as is shown in FIG. 10, the implant 6 is in the form of a patch or scaffold. Such a patch or scaffold is in, for example, the form of a biological material. For example, an extracellular matrix in the form of, for example, an SIS patch. Such a patch is provided by DePuy Orthopaedics, Inc., Warsaw, Ind., in the trade name of the Restore® patch. The implant 6 has a shape or size similar to that of the glenoid fossa and is positioned over the glenoid fossa to receive the suture anchors, for example, first suture anchor 16, second suture anchor 18, third suture anchor 20 and fourth suture anchor 22. The suture anchor 16 may include suture pairs attached to the respective suture anchors. For example, the first suture anchor 16 may include a first suture anchor pair 89, the second suture anchor 18 may include a second suture pair 91. Similarly, the third suture anchor 20 may include a third suture pair 93. Similarly, the fourth suture anchor 22 may include a fourth suture pair 95.

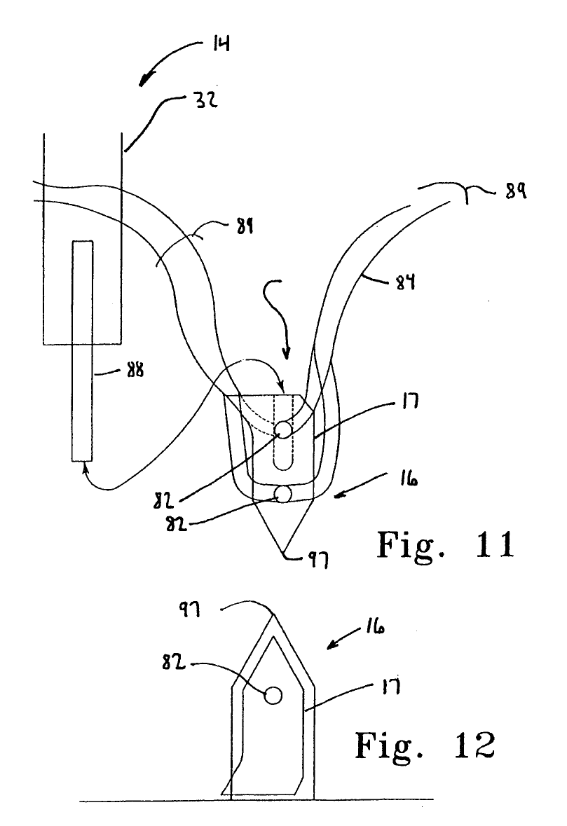

Referring now to FIGS. 11 and 12, the first suture anchor 16 is shown in greater detail. The first suture anchor 16 includes a body 17 defining suture holes 82 for receiving sutures 84. The sutures 84 may form a suture pair or group 89. The body 17 further defines the cavity 86, which may have a generally cylindrical shape.

The cavity 86 is adapted to slidably receive stem 88 extending from the first column 32. The slidable fit of the stem 88 to the cavity 86 provides for the release of the suture anchor 16 from the cartridge 14 when the suture anchor 16 engages the glenoid fossa. The suture anchor 16 may include a cutting edge 97 along the periphery of the body 17 of the first suture anchor 16 for engaging the glenoid fossa.

Referring now to FIG. 13, the suture anchor delivery device 10 is shown in position by the patient being installed in the direction of arrow 99. The suture anchors are then ready to be deployed either directly into glenoid fossa 8 of the scapula 7 or alternatively through the implant 6 and into glenoid fossa 8 of the scapula 7.

Referring now to FIG. 13A, the implant 6 is shown in greater detail. The implant 6 has a generally oval shape and a shape that conforms to the general shape of the glenoid fossa, in that it is to be implanted against the glenoid fossa. The implant 6 may be in the form of a biological material having a layer or a plurality of layers in forming a sheet. The implant 6 may be in the form of, for example, a vertebral extracellular matrix product, for example, a SIS processed material. Such material is more fully described in U.S. Pat. Nos. 4,902,508; 4,956,178; 5,372,821; and 5,955,110 and assigned to Purdue Research Corporation. Such a product is provided by DePuy Orthopaedics, Inc. and sold as the Restore® patch.

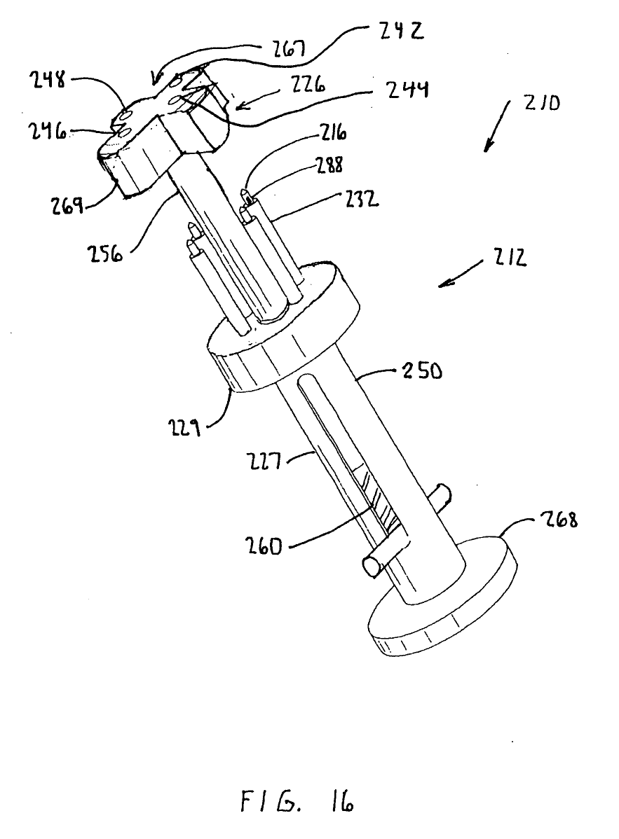

Referring now to FIG. 14, the Restore® patch 6 is shown positioned in the glenoid cavity 8 of the scapula 7. The Restore® patch 6 is secured to the glenoid fossa 8 of the scapula 7 by, for example, the four spaced apart suture anchors. For example, the first suture anchor 16, the second suture anchor 18, the third suture anchor 20 and the fourth suture anchor 22. The Restore® patch 6 is further secured to the glenoid cavity 8 through the use of sutures 84 that are secured to the anchors 16, 18, 20 and 22.

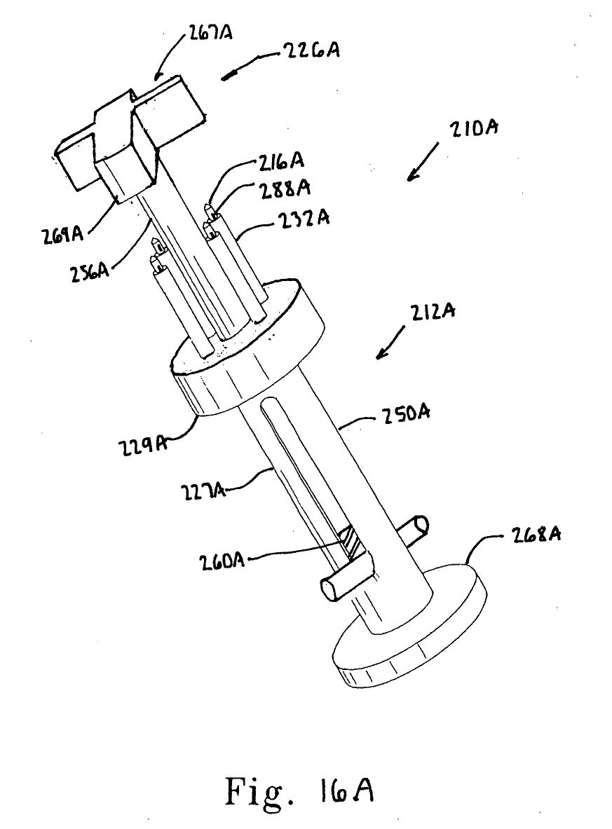

While the present invention may be practiced with a cartridge, it should be appreciated that the suture anchor delivery device of the present invention may have embodiments, including those with cartridges. It should be appreciated that within the scope of the present invention, a suture anchor delivery device for delivering a plurality of sutures may be provided without a cartridge. For example, and referring now to FIG. 15, yet another embodiment of the present invention is shown as suture anchor delivery device 110. The suture anchor delivery device 110 is utilized for performing surgery on tissues of a patient. The suture anchor delivery device 110 includes an applicator 112, as well as a plurality of suture anchors 116. Each of the suture anchors 116 is operatively associated with the applicator 112. The applicator 112 as shown in FIG. 15, includes a body 150.

The body 150 may, as is shown in FIG. 15, include a central body position 127, as well as a base 168 extending from a first end of the central body portion 127 and a head 129 extending from the opposed end of the central body portion 127. The suture anchors 116 are removably secured to the head 129 of the applicator 112. For example, the head 129 may include a plurality of stems 188 positioned on the surface of the head 129. Each of the suture anchors 116 matingly receives one of the stems 188, which provides for a removable securement of the suture anchors 116 to the applicator 112. The applicator 112 is positioned against the glenoid fossa with the suture anchors 116 positioned over the implant. The implant is positioned between the suture anchors 116 and the glenoid fossa. The base 150 is struck with, for example, a mallet, and the suture anchors 116 are released from the applicator 112.

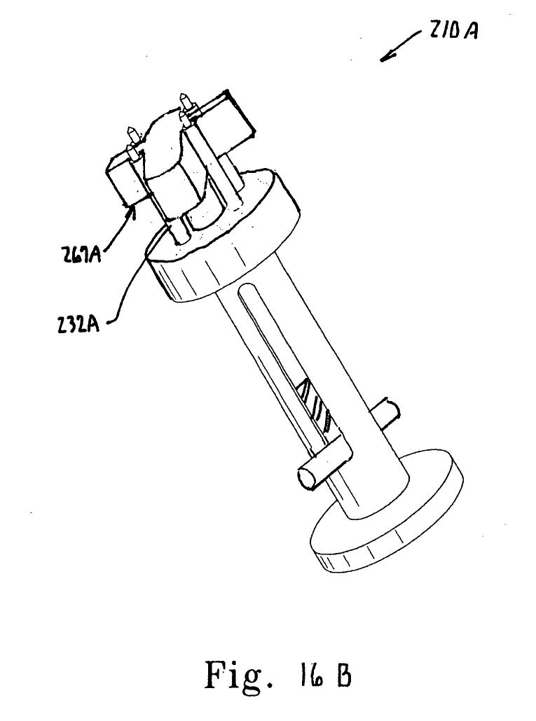

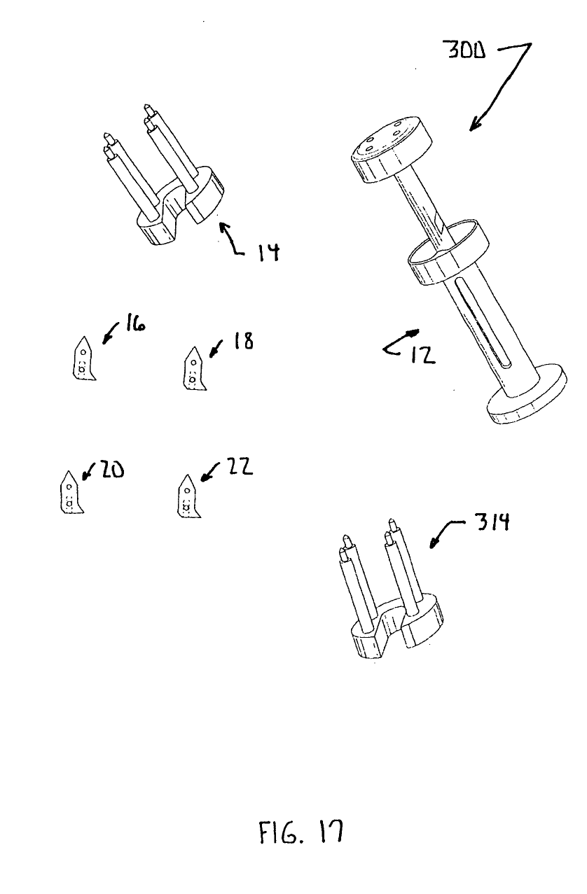

Referring now to FIG. 16, yet another embodiment of the present invention is shown as suture anchor delivery device 210. The suture anchor delivery device 210 of FIG. 16, like the suture anchor delivery device 110 of FIG. 15, does not include a cartridge. The suture anchor delivery device 210 includes a applicator 212. The applicator 212 is utilized to receive a plurality of suture anchors 216.

The applicator 212 includes a body 250. The body 250 includes a tubular portion 227. Extending from the tubular portion 227 is a body base 268. Opposed to the body base 268 is a head 229. A plurality of columns 232 extend outwardly and in parallel from the head 229. Each of the columns 232 include a stem 288 for receiving the suture anchors 216. The stems 288 provide for a removable securement of the suture anchors 216. The applicator 212 further includes a guide 226 for cooperation with the glenoid fossa. The guide 226 is movably oriented axially.

The guide 226, as is shown in FIG. 16, is connected to a plunger shaft 256. The plunger shaft 256 is moveably fitted in the tubular portion 227 of the body 250. A spring 260 is utilized to urge the guide 226 away from the body 250. Relief areas 267 are formed in the guide 226 for receiving the columns 232 and the suture anchors 216, which are attached to the columns 232.

When utilizing the suture anchor delivery device 210 of FIG. 16, the guide 226 is positioned against the implant and the glenoid fossa. The body base 268 is advanced toward the glenoid fossa, causing the suture anchors 216 and the columns 232 to advance through the openings 242, 244, 246, and 248 formed in lobes 269. Relief areas 267 of the guide 226 permit viewing of the suture anchors 216 as they engage in the glenoid fossa.

Referring now to FIG. 16A, yet another embodiment of the present invention is shown as suture anchor delivery device 210A. The suture anchor delivery device 210A of FIG. 16A, like the suture anchor delivery device 210 of FIG. 16, does not include a cartridge. The suture anchor delivery device 210A includes a applicator 212A. The applicator 212A is utilized to receive a plurality of suture anchors 216A.

The applicator 212A includes a body 250A. The body 250A includes a tubular portion 227A. Extending from the tubular portion 227A is a body base 268A. Opposed to the body base 268A is a head 229A. A plurality of columns 232A extend outwardly and in parallel from the head 229A. Each of the columns 232A include a stem 288A for receiving the suture anchors 216A. The stems 288A provide for a removable securement of the suture anchors 216A. The applicator 212A further includes a guide 226A for cooperation with the glenoid fossa. The guide 226A is movably oriented axially.

The guide 226A, as is shown in FIG. 16A, is connected to a plunger shaft 256A. The plunger shaft 256A is moveably fitted in the tubular portion 227A of the body 250A. A spring 260A is utilized to urge the guide 226A away from the body 250A. Relief areas 267A are formed in the guide 226A between lobes 269A for receiving the columns 232A and the suture anchors 216A, which are attached to the columns 232A.

When utilizing the suture anchor delivery device 210A of FIG. 16A, the guide 226A is positioned against the implant and the glenoid fossa. The body base 268A is advanced toward the glenoid fossa, causing the suture anchors 216A and the columns 232A to advance through the relief areas 267A of the guide 226A and permitting the suture anchors 216A to engage in the glenoid fossa. The relief areas aid in viewing the columns 232A and the suture anchors 216A as they engage the glenoid.

Referring now to FIG. 16B, the suture anchor delivery device 210A of FIG. 16A is shown in its contracted position. In this position the columns 232A are show in position in the relief portions 267A.

Referring now to FIG. 17, yet another embodiment of the present invention is shown as kit 300 for installing suture anchors to a patient. The suture anchor delivery kit includes the applicator 12 of FIG. 4 as well as the cartridge 14 of FIG. 2. The suture anchor delivery kit further includes the anchors as shown in FIG. 2, for example, the first anchor 16, the second anchor 18, the third anchor 20, and the fourth anchor 22. The suture anchor delivery kit 300 may further include a second cartridge 314 similar to the first cartridge 14. The second cartridge 314 may be utilized after the first cartridge 314 has been utilized.

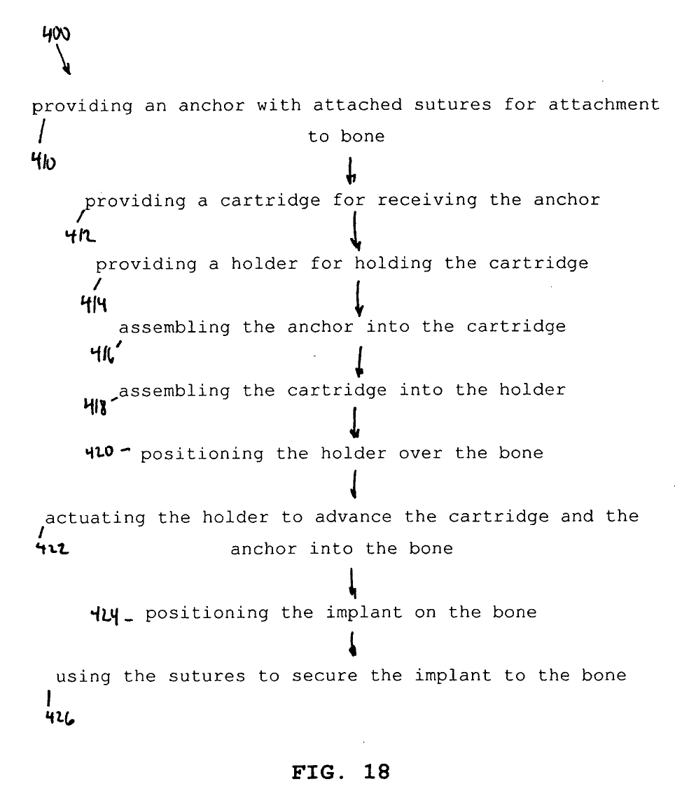

Referring now to FIG. 18, yet another embodiment of the present invention is shown as surgical procedure or surgical method 400. The surgical procedure 400 is for use in securing surgical suture anchors into a patient. The method 400 includes a first step 410 of providing an anchor with attached sutures for attachment to bone. The method 400 further includes a second step 412 of providing a cartridge for receiving the anchor. The method 400 further includes a third step 414 of providing a holder for holding the cartridge as well as a fourth step 416 of assembling the anchor into the cartridge. The method 400 further includes a fifth step 418 of assembling the cartridge into the holder. The method 400 further includes a sixth step 420 of positioning the holder over the bone and an seventh step 422 of actuating the holder to advance the cartridge and the anchor into the bone. The method 400 further includes a eighth step 424 of positioned the implant on the bone and a ninth step 426 of using the sutures to secure the implant to the bone. It should be appreciated the steps of the method 400 may be reordered as the surgeon deems advisable. For example the eighth step 424 of positioning the implant on the bone may be performed before the sixth step 418 of positioning the holder over the bone.

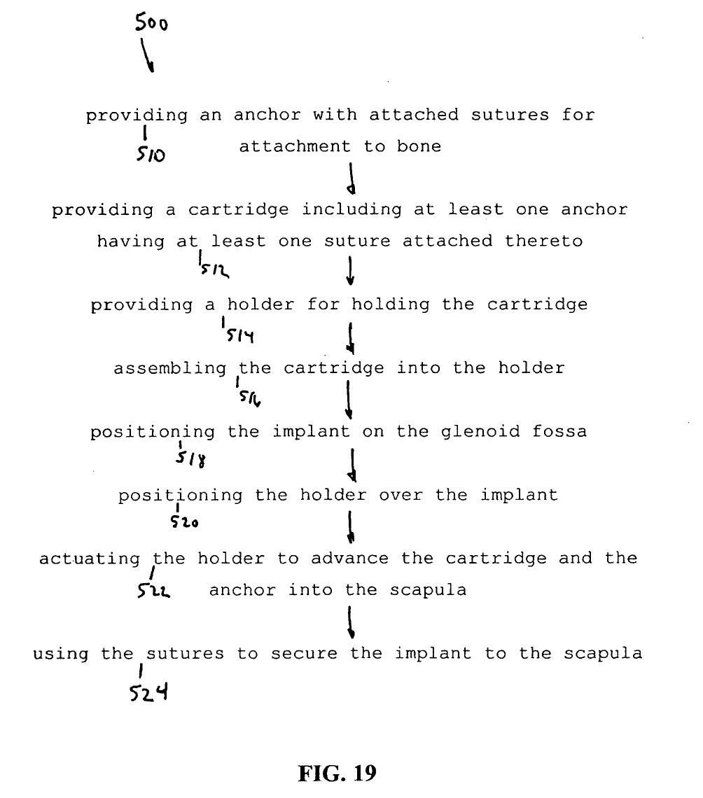

Referring now to FIG. 19, yet another embodiment of the present invention is shown as surgical procedure 500. The surgical procedure 500 is for implanting a suture anchor into a patient. The method 500 includes a first step 510 of providing an anchor with attached sutures for attachment to bone. The method 500 further includes a second step 512 of providing a cartridge, including at least one anchor having at least one suture attached to the anchor. The method 500 further includes a third step 514 of providing a holder for holding the cartridge, and a fourth step 516 of assembling the cartridge into the holder. The method 500 further includes a fifth step 518 of positioning the implant on the glenoid fossa and a sixth step 520 of positioning the holder over the implant. The method 500 further includes a seventh step 522 of actuating the holder to advance the cartridge and the anchor into the scapula. The method 500 further includes an eighth step 524 of utilizing the sutures to secure the implant to the scapula. It should be appreciated the steps of the method 500 may be reordered as the surgeon deems advisable. For example the fifth step 518 of positioning the implant on the glenoid fossa may be performed after the seventh step 522 of actuating the holder to advance the cartridge and the anchor into the scapula.

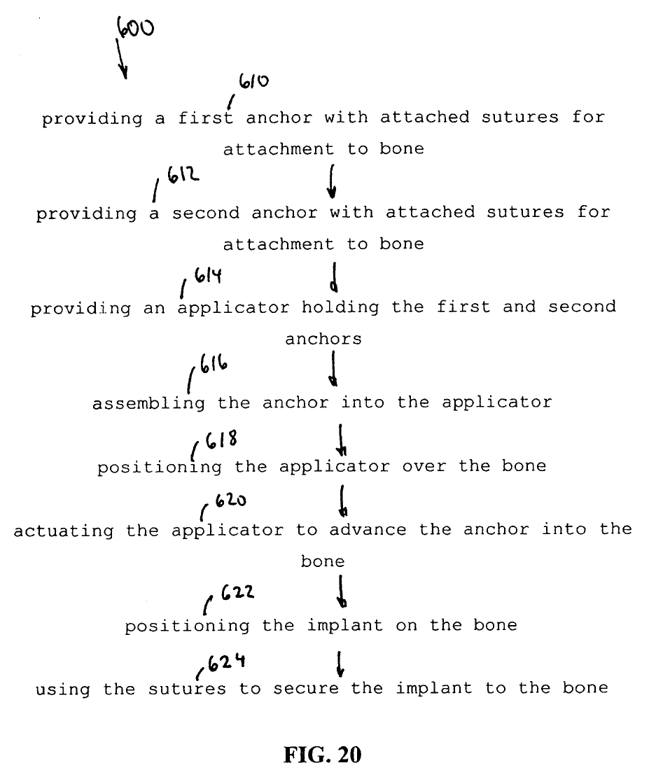

Referring now to FIG. 20, yet another embodiment of the present invention is shown as surgical procedure or surgical method 600. The surgical procedure 600 is for implanting a suture anchor into a patient. The method 600 includes a first step 610 of providing a first anchor with attached sutures for attachment to bone. The method 600 includes a second step 612 of providing a second anchor with attached sutures for attachment to bone and a third step 614 of providing an applicator holding the first and second anchors.

The method 600 further includes a fourth stem 616 of assembling the anchor into the applicator and a sixth step 618 of positioning the applicator over the bone. The method 600 also includes a seventh step 620 of actuating the applicator to advance the anchor into the bone and an eighth step 622 of positioning the implant on the bone. The method 600 also includes a ninth step 624 of using the sutures to secure the implant to the bone. It should be appreciated the steps of the method 600 may be reordered as the surgeon deems advisable. For example the eighth step 622 of positioning the implant on the bone may be performed before the sixth step 618 of positioning the applicator over the bone.

Although the present invention and its advantages have been described in detail, it should be understood that various changes, substitutions, and alterations can be made therein without departing from the spirit and scope of the present invention as defined by the appended claims.

We claim:

1. A suture anchor delivery device for use in performing surgery on tissue of a patient, said suture anchor delivery device comprising:
an applicator;
a cartridge, removably secured to said applicator; and
an anchor operatively associated with said cartridge.

2. The suture anchor delivery device of claim 1, wherein said anchor comprises:
a connecting portion for connecting said anchor to said applicator; and
a suture portion connected to the connecting portion.

3. The suture anchor delivery device of claim 1, further comprising a second anchor operatively associated with said cartridge and spaced from said first mentioned anchor.

4. The suture anchor delivery device of claim 3, further comprising a third anchor operatively associated with said cartridge and spaced from said first mentioned anchor and from said second anchor.

5. The suture anchor delivery device of claim 4, further comprising a fourth anchor operatively associated with said cartridge and spaced from said first anchor, from said second anchor and from said third anchor.

6. The suture anchor delivery device of claim 4, wherein said first anchor, said second anchor, and said third anchor are spaced from each other in a pre-elected pattern.

7. The suture anchor delivery device of claim 1, wherein said applicator comprises a portion thereof for cooperation with the tissue of the patient.

8. The suture anchor delivery device of claim 1, further comprising a guide operatively associated with said applicator and with said cartridge.

9. The suture anchor delivery device of claim 8, wherein said guide defines a surface thereof closely conforming to the tissue of the patient.

10. The suture anchor delivery device of claim 8, wherein said guide is slidably connected to said applicator

11. The suture anchor delivery device of claim 8:
wherein said guide defines a surface for engagement with the tissue of the patient
wherein said guide, said cartridge and said applicator are configured to have a first relationship in which a portion of said cartridge extends beyond the surface of said guide and a second relationship in which said cartridge is positioned below surface of said guide

12. The suture anchor delivery device of claim 11:
further comprising biasing means; and
wherein said biasing means biased said cartridge in the second relationship.

13. The suture anchor delivery device of claim 12, wherein said biasing means comprises a spring

14. The suture anchor delivery device of claim 11, wherein said biasing means comprises a spring.

15. The suture anchor delivery device of claim 11:
wherein said cartridge includes a stem for receiving said anchor; and
wherein said guide includes an internal wall defining an opening for the passage of at least a portion of said stem there through;

16. The suture anchor delivery device of claim 8:
wherein said guide defines a surface thereof for cooperation with the tissue of said patient; and
wherein a portion of said surface defines a recessed face.

17. The suture anchor delivery device of claim 1, wherein said applicator defines a cavity thereof for receiving at least a portion of said cartridge.

18. The suture anchor delivery device of claim 1, wherein said applicator defines a surface thereof opposed to said cartridge for use in exerting force in the direction of said anchor to urge said anchor into the soft tissue.

19. A anchor delivery kit for use in performing surgery on tissue of a patient, said anchor delivery kit comprising:
a suture applicator
a cartridge, removably securable to said applicator; and
an anchor removably securable to said cartridge.

20. A method for securing an implant onto a glenoid fossa of a scapula comprising the steps of:
providing an anchor with attached sutures for attachment to bone;
providing a cartridge for receiving the anchor;
providing a holder for holding the cartridge;
assembling the anchor into the cartridge;
assembling the cartridge into the holder;
positioning the holder over the bone;
actuating the holder to advance the cartridge and the anchor into the bone;
positioning the implant on the bone; and
using the sutures to secure the implant to the bone.

21. The method of securing the implant as in claim 20, wherein the step of positioning the implant is performed before the step of actuating the holder.
